(19) United States
(12) Patent Application Publication
(10) Pub. No.: US 2024/0390413 A1
(43) Pub. Date: Nov. 28, 2024

(54) POLYMERS OBTAINED BY THE REACTION OF A PHENOLIC COMPOUND AND A CARBONYL COMPOUND AS ANTI-SARS-COV-2 AGENTS

(71) Applicants: BASF SE, Ludwigshafen (DE); CHRISTIAN-ALBRECHTS-UNIVERSITÄT ZU KIEL, Kiel (DE); DEUTSCHES PRIMATENZENTRUM GMBH, Göttingen (DE)

(72) Inventors: Guenter Scherr, Ludwigshafen (DE); Sandra Gloria König, Ludwigshafen (DE); Ulrich Mrowietz, Kiel (DE); Stefan Poehlmann, Göttingen (DE)

(21) Appl. No.: 18/696,406

(22) PCT Filed: Sep. 29, 2022
(86) PCT No.: PCT/EP2022/077137

§ 371 Date: Mar. 28, 2024

(30) Foreign Application Priority Data
Sep. 30, 2021 (WO) PCT/EP2021/077007

(51) Int. Cl. A61K 31/795; A61K 31/245; A61K 31/675; A61K 39/42; A61P 31/14; C08G 14/08

(52) U.S. Cl. CPC A61K 31/795; A61K 31/245; A61K 31/675; A61K 39/42; A61P 31/14; C08G 14/08

(57) ABSTRACT

The present invention relates to a condensation product, obtained or obtainable by the reaction of phenol, formaldehyde, sulfuric acid and urea, or a pharmaceutical composition comprising the same for use in a method for the prevention or treatment of Coronavirus disease 2019 (COVID-19) and/or for the prevention or treatment of severe acute respiratory syndrome coronavirus 2 (SARS-COV-2) infection, to a pharmaceutical set comprising such a pharmaceutical composition for the same use and to such a pharmaceutical composition or pharmaceutical set.

The present invention relates to a condensation product or a pharmaceutical composition comprising the same for use in a method for the prevention or treatment of Coronavirus disease 2019 (COVID-19) and/or forth prevention or treatment of severe acute respiratory syndrome coronavirus 2 (SARS-CoV-2) infection, to a pharmaceutical set comprising such a pharmaceutical composition for the same use and to such a pharmaceutical composition or pharmaceutical set.

COVID-19 is a highly contagious disease. Its diverse symptoms include breathing difficulties, headache, loss of smell and taste, fever, nasal congestion, and muscle pain amongst others, with their intensity ranging from mild symptoms to severe illness.

The first case of COVID-19 was identified in December 2019 and the disease has since spread rapidly worldwide. COVID-19 is caused by an infection with SARS-CoV-2.

SARS-CoV-2 is a member of the Coronaviridae family, of the genus Betacoronavirus and containing a positive-sense single-stranded RNA (+ssRNA) genome of about 30,000 bases. There are several known variants of SARS-CoV-2, some of which bear the potential for increased transmissibility and virulence.

In general, coronaviruses (CoVs) are classified into four major genera: Alphacoronavirus, Betacoronavirus (which primarily infect mammals), Gammacoronavirus, and Deltacoronavirus (which primarily infect birds). In addition to SARS-CoV-2, the genus Betacoronavirus further includes SARS-CoV-1 and MERS-CoV. SARS-CoV-1 is the closest relative to SARS-CoV-2.

SARS-CoV-2 is unique among known betacoronaviruses in its incorporation of a polybasic cleavage site, a characteristic known to increase pathogenicity and transmissibility in other viruses. Particularly, the spike protein of the virus may cause sufficient affinity to the angiotensin converting enzyme 2 (ACE2) receptors of human cells to use them as a mechanism of cell entry. Further, SARS-CoV-2 has usually a higher affinity to human ACE2 than SARS-CoV-1.

The genome of coronaviruses typically contains a +ssRNA but it differs in size ranging between approximately 26 and 32 kb. It also includes a variable number of open reading frames (ORFs). The first ORF is the largest, encoding nearly 70% of the entire genome and 16 non-structural proteins (nsps). Of the nsps, the main protease (Mpro, also known as a chymotrypsin-like cysteine protease, 3CLpro; preferably NCBI Reference Sequence: YP_009742612.1), encoded by nsp5, has been found to play a fundamental role in viral gene expression and replication, and thus it is an attractive target for anti-CoV drug design.

Since it plays such a fundamental role in processing the polyproteins that are translated from the viral RNA, Mpro has been suggested as an important drug target for SARS-CoV-2 infections. Furthermore, since Mpro plays such an essential role in SARS-CoV-2, it is suggested that a drug targeting SARS-CoV-2 Mpro will also be useful against the different SARS-CoV-2 variants, particularly since most mutations affect the spike-protein of SARS-CoV-2 and do not affect Mpro. Thus, finding a drug targeting SARS-CoV-2 Mpro may also provide the advantage that the drug is useful even in case further SARS-CoV-2 variants emerge.

However, difficulties have been observed in finding a drug, which may effectively target the SARS-CoV-2 Mpro and which may thus provide an efficient treatment for SARS-CoV-2 infection and COVID-19 for the present—and future—variants of SARS-CoV-2.

These difficulties may be explained by the fact that the Mpro enzymes of SARS-CoV-1 and SARS-CoV-2 show major differences in shape and size, which indicates that drugs proven useful against SARS-CoV-1 do not show an effect against SARS-CoV-2. This is supported by the fact that even though drugs against SARS-CoV-1 are known, finding a drug against SARS-CoV-2 has currently been of limited success.

Bzowka et al., “Structural and Evolutionary Analysis Indicate That the SARS-CoV-2 Mpro Is a Challenging Target for Small-Molecule Inhibitor Design”, Int. J. Mol. Sci., 2020, 21, 3099 discloses that in spite of a high level of sequence similarity, the active sites in the Mpro proteins of SARS-CoV-1 and SARS-CoV-2 showed major differences in both shape and size, indicating that repurposing known SARS drugs for COVID-19 may be futile.

In addition to the molecular level, SARS-CoV-1 and SARS-CoV-2 also differ in their main localization in the human body. Whereas SARS-CoV-2 mainly reproduces in the upper throat and thus causes a high viral load in this region, SARS-CoV-1, which caused a pandemic of the disease SARS in 2003, generally does not reproduce in the upper throat but mainly reproduces in the lower parts of the lung. Thus, with a high viral load only in lower parts of the lung, the spread of SARS-CoV-1 is significantly lower than SARS-CoV-2. Consequently, prevention and/or therapy approaches with regard to SARS-CoV-2 need to differ from those targeting SARS-CoV-1.

As of August 2021, the global COVID-19 pandemic has caused more than 4 million deaths as a result of over 200 million confirmed cases (https://covid19.who.int/) generating an extensive demand for effective treatment and prevention strategies for COVID-19 as well as for the underlying SARS-CoV-2 infections.

Several treatment options for viral infections exist. In general, antiviral treatment can be based on virostatic effects, i.e. the inhibition of virus reproduction, or on virucidal effects, i.e. the deactivation or destruction of viruses or viral particles. Both effects—separately or in combination—may effectively prevent a progression of the infection and the corresponding disease.

Presently, several vaccinations against SARS-CoV-2 have been developed. However, the newly developed vaccinations may not be applied to any person. For instance, certain groups of the population cannot be vaccinated (e.g. due to medical predispositions or pregnancy) or a vaccination is met with certain reservation and thus refused. Moreover, in contrast to industrialized countries, many countries do not have access to a sufficient amount of vaccine doses, since the production is cost-expensive and the produced vaccine doses are mostly distributed over the industrialized countries. Thus, there is a need for affordable and widely-accessible prevention and/or treatment options for SARS-CoV-2 infection.

Up to now, promising candidates for successfully treating and/or preventing COVID-19 and/or a SARS-CoV-2 infection are very limited and most of the previously known antiviral agents have been proven insufficient in providing an antiviral effect against SARS-CoV-2, which supports the findings of Bzowka et al., as reported above.

Generally, for treating viral infections, several condensation products have been previously tested. However, an antiviral effect against SARS-CoV-2 has not been disclosed or suggested.

WO 2007/131811 describes a mixture comprising one or more condensation products and at least one tanning agent, its production, and its use as a pharmaceutical agent or disinfectant. The mixture comprises a condensation product with an M_{w} of ≥9,000 g/mol as well as at least one tanning agent with an M_{w} of ≤3,000 g/mol. The disclosed mixtures show antiviral activity i.e. against herpes simplex virus and Human papillomavirus, which are both DNA-viruses, as well as inhibitory properties against human leukocyte elastase (HLE). An inhibitory effect against herpes simplex virus is observed in Vero cell cultures but is limited to the case in which mixture and virus suspension were added simultaneously. However, no effect with regard to SARS-CoV-2 is shown in WO 2007/131811.

WO 2007/131812 describes the use of tanning agents in or on filters, e.g. as components of respirator masks, in order to avoid infections by decreasing and/or eliminating the overall amount of virus particles or bacterial cells in inhaled air. WO 2007/131812 focusses on decreasing the amount of avian influenza A virus particles and does not mention coronaviruses. Furthermore, WO 2007/131812 describes the use of the tanning agents in a respiratory device, whereas no administration to the organism occurs.

DE 10 2004 034 613 A1 discloses the pharmaceutical use of condensation products, which were previously separated according to their molecular size. The respective condensation products with an M_{w} in the range from 1,000 to 100.00 g/mol shall serve as general antiviral agents, in particular herpesviruses, i.e. Human alphaherpesvirus and Variecella zoster virus, which are both DNA-viruses. Furthermore, an effect is only demonstrated in Vero cell cultures and is limited to the case in which the condensation products and virus suspension were added simultaneously.

WO 95/14479 describes the use of condensation polymers as a therapeutic agent inhibiting CD4/gp120 binding in individuals infected with the human immunodeficiency virus (HIV) and thus inhibiting HIV infectivity of CD4+ cells and HIV-induced syncytia formation. Thus, the condensations polymers are described to be used for treatment of acquired immunodeficiency syndrome (AIDS), AIDS-related complex (ARC), AIDS-related dementia, and non-symptomatic HIV infections. The average molecular weight (MW) for all given examples ranges from 2,000 to 56,000 g/mol. However, coronaviruses are not mentioned in WO 95/14479.

U.S. Pat. No. 4,604,404 describes the use of condensation polymers as antiviral agents against herpesviruses, i.e. Human alphaherpesvirus type 1 and 2, which are DNA-viruses. The treatment is described as a topical application. The condensation polymers have a molecular weight ranging from 500 to 10,000 g/mol. However, coronaviruses are not mentioned in U.S. Pat. No. 4,604,404.

None of the above-mentioned prior art discloses the use of respective condensation products or pharmaceutical compositions comprising the same in a method for the prevention or treatment of COVID-19 and/or for the prevention or treatment of a SARS-CoV-2 infection. Furthermore, and as described above, even potential treatment options for SARS-CoV-1 infections are considered to be useless against SARS-CoV-2, even more against possible further variants of SARS-CoV-2. Therefore, there is a strong need for options to fight the present SARS-CoV-2 pandemic, particularly in light of possible further variants of SARS-CoV-2, which may evade current vaccines or the few possible drugs against SARS-CoV-2.

Therefore, the primary object of the present invention was to provide possibilities, preferably long-lasting possibilities, for the prevention or treatment of COVID-19 and/or for the prevention or treatment of a SARS-CoV-2 infection. Preferably, the provided possibilities shall be cost-efficient and thus be easily accessible, also for non-industrialized countries.

The primary object is solved by a condensation product or a salt thereof or a pharmaceutical composition comprising the condensation product or one or more salt(s) thereof or the condensation product and one or more salt(s) thereof,
wherein the condensation product is obtained or obtainable by reaction of
a1) at least one phenolic compound, and
a2) at least one carbonyl compound,
a3) and optionally: at least one sulfonating agent,
a4) and optionally: urea and/or one or more urea derivative(s),
wherein the condensation product or salt thereof has a molecular weight M_{w} in the range of from 500 g/mol to 50,000 g/mol, preferably in the range of from 500 g/mol to 5,000 g/mol,
for use in a method for the prevention or treatment of COVID-19 and/or for the prevention or treatment of a SARS-CoV-2 infection,
wherein the method comprises the step of administering the condensation product or a salt thereof or the condensation product and a salt thereof to a subject, preferably a human,
wherein the prevention of COVID-19 comprises the step of administering to a subject the condensation product or a salt thereof or the condensation product and a salt thereof, wherein a first administration is performed not later than 3 months after a positive diagnosis of the SARS-CoV-2 infection of the subject, and
wherein the treatment of COVID-19 comprises the step of administering to a subject the condensation product or a salt thereof or the condensation product and a salt thereof, wherein a first administration is performed not later than 3 months after a positive diagnosis of the SARS-CoV-2 infection of the subject, and
wherein the treatment of a SARS-CoV-2 infection comprises the step of administering to a subject the condensation product or a salt thereof or the condensation product and a salt thereof, wherein a first administration is performed not later than 3 months after a positive diagnosis of the SARS-CoV-2 infection of the subject,
preferably wherein the pharmaceutical composition further comprises one or more pharmaceutically acceptable carrier(s).

Surprisingly it was found that the condensation product as described herein or a salt thereof can be used in a method for the prevention or treatment of COVID-19 and/or for the prevention or treatment of a SARS-CoV-2 infection and it surprisingly shows a pronounced antiviral activity against SARS-CoV-2 as compared to SARS-CoV-1.

Even more surprisingly, it was found that the condensation product as described herein or a salt thereof seems to target the SARS-CoV-2 Mpro and thus provides the advantages of a long-lasting possibility for fighting the SARS-CoV-2 pandemic, as described above. Particularly, the condensation product as described herein or a salt thereof can be used against all—or at least most of—the SARS-CoV-2 variants. It is therefore preferred in the prevention and/or treatment of COVID-19 and/or the prevention and/or treatment of a SARS-CoV-2 infection, that the method comprises the reduction of SARS-CoV-2 Mpro activity. Particularly, it is preferred in the treatment of COVID-19 and/or in the treatment of a SARS-CoV-2 infection, that the method comprises the reduction of SARS-CoV-2 Mpro activity. Additionally or alternatively, it is preferred in the prevention of COVID-19 and/or in the treatment of a SARS-CoV-2 infection, that the method comprises the reduction of SARS-CoV-2 Mpro activity. Additionally or alternatively, it is preferred in the treatment and/or prevention of COVID-19 and/or in the treatment of a SARS-CoV-2 infection, that the method comprises the reduction of SARS-CoV-2 Mpro activity.

With regard to the condensation product, component a1) may, for instance, be selected from the group consisting of phenol, ortho-cresol, meta-cresol, para-cresol, 2-ethylphenol, 3-ethylphenol, 4-ethylphenol, 2,3-5 dimethylphenol, 2,4-dimethylphenol, 2,5-dimethylphenol, 2,6-dimethylphenol, 3,4-dimethylphenol, 3,5-dimethylphenol, gallic acid, 9-hydroxyanthracene as a tautomer of anthrone, 9-hydroxyphenanthrene, diphenylmethane, phenyl-(2-methylphenyl)methane, phenylparatolylmethane, phenylmetatolylmethane.

Additionally or alternatively, component a2) may be selected from the group consisting of aldehydes and ketones, such as formaldehyde, acetaldehyde or propionaldehyde.

Additionally or alternatively, component a3) may be selected from the group consisting of sulphuric acid, chlorosulphonic acid, amidosulphonic acid, and oleum having an SO₃ content in the range of from 1 to 30% by weight.

Additionally or alternatively, component a4) may be selected from the group consisting of urea and derivatives thereof. Preferably, component a4) is selected from the group consisting of urea and a derivative thereof, wherein the derivative has a hydrogen atom on each nitrogen atom.

Advantageously, the condensation product according to the present invention or a salt thereof is easy to produce, including its production in large amounts while its production costs are relatively low in comparison to other pharmaceutical agents. Thus, it might be comparatively easy to ensure its global distribution, especially its provision also for developing regions.

The term “M_{w}” as used herein refers to the weight-average molecular weight as determined by gel permeation chromatography (GPC) standard procedures, DIN standard 55672-1, using tetrahydrofuran (THF) as a solvent. Preferably, the weight-average molecular weight is determined by GPC with the following parameters:
Stationary phase: poly (2-hydroxymethacrylate) gel cross-linked with ethylene glycol dimethacrylate (commercially available as HEMA BIO from PSS, Mainz, Germany);
Eluent: Mixture of 30% by weight of tetrahydrofuran (THF), 10% by weight of acrylonitrile, 60% by weight of 1-molar NaNO3 solution, each related to the total weight of the mixture;
Internal standard: 0.001% by weight of benzophenone, based on the mobile phase;
Flow: 1.5 ml/min;
Concentration: 1% by weight, related to the eluent with the internal standard, in the eluent with the internal standard;
Detection: UV/Vis spectrometry at 254 nm;
Calibration with polystyrene calibration part of the company PSS.

The term “SARS-CoV-2” as used herein refers to severe acute respiratory syndrome coronavirus 2, which is a member of the Coronaviridae family, and of the genus Betacoronavirus.

The term “COVID-19” as used herein refers to a disease caused by an infection with SARS-CoV-2, causing symptoms such as breathing difficulties, headache, loss of smell and/or taste, fever, nasal congestion, and/or muscle pain, with their intensity ranging from mild symptoms to severe illness up to death. Whether these symptoms are considered as symptoms of COVID-19 or not is preferably determined by verifying a SARS-CoV-2 infection, as described herein, wherein a positive SARS-CoV-2 infection determines that the symptoms are symptoms of COVID-19.

Preferably, the prevention of COVID-19 also comprises the prevention of a SARS-CoV-2 infection.

Preferably, the treatment of COVID-19 also comprises reducing the infectivity of a subject, which may be represented by or which may include reducing the viral load in the subject.

The term “infection” as used herein refers to the condition immediately after a virus has invaded an organism and has entered a cell of the organism. Preferably, the term “infection” describes the condition that a virus has replicated within the organism. Preferably, the term “infection” refers to a detectable infection, i.e. the condition, in which a virus has already replicated within the organism and the SARS-CoV-2 infection can be diagnosed, as described herein.

A method for the prevention or treatment of COVID-19 and/or for the prevention or treatment of a SARS-CoV-2 infection preferably comprises causing a virostatic and/or virucidal effect against SARS-CoV-2.

The term “virostatic” as used herein preferably refers to an inhibitory effect on viral replication, wherein the inhibitory effect may include an impairment or the complete inhibition of the viral replication.

The term “virucidal” as used herein refers to an inhibition or destruction of the virus.

Furthermore, it is preferred that the prevention of COVID-19 comprises the step of administering to a subject the condensation product or a salt thereof or the condensation product and a salt thereof before an infection with SARS-CoV-2.

The administration of the condensation product or a salt thereof or the condensation product and a salt thereof for the treatment or prevention of COVID-19 and/or for the treatment, and preferably for the prevention, of a SARS-CoV-2 infection according to the present invention is performed no later than three months (i.e. 12 weeks), preferably no later than two and a half months (i.e. 10 weeks), preferably no later than two months (i.e. 8 weeks), more preferably no later than one and a half months (i.e. 6 weeks), preferably no later than one month (i.e. 4 weeks), preferably no later than three weeks, preferably no later than two weeks, preferably no later than 13 days, preferably no later than 12 days, preferably no later than 11 days, preferably no later than 10 days, preferably no later than 9 days, preferably no later than 8 days, preferably no later than 7 days, preferably no later than 6 days, preferably no later than 5 days, preferably no later than 4 days, preferably no later than 3 days, preferably no later than 2 days, preferably no later than 1 day, after a positive diagnosis of the SARS-CoV-2 infection in the subject.

The diagnosis of a SARS-CoV-2 infection may be achieved via known methods. Such methods may include antigen tests, preferably a commercially available antigen test, polymerase chain reaction (PCR) analysis, such as reverse transcription PCR (RT-PCR) analysis and/or quantitative RT-PCR (qRT-PCR) analysis and combinations thereof. Preferably, the diagnosis of a SARS-CoV-2 infection is achieved by a method comprising at least PCR analysis, preferably at least RT-PCR analysis, particularly preferably at least qRT-PCR analysis. Particularly preferably, the method does not comprise a SARS-CoV-2 antibody test.

Preferably, pharmaceutically acceptable carriers, as described herein, may be unpolar substances, preferably selected from the group consisting of polyacrylates, polymethacrylates, polyethers, polyesters, polyamides, polynitriles, etherificated celluloses, celluloseacetate, polynitrile, etherificated sugars and mixtures thereof.

Furthermore, the pharmaceutical composition may also comprise one or more additives, preferably selected from the group consisting of fillers, binders, lubricants, wetting agents, stabilizers, surfactants, preferably ionic surfactants and/or non-ionic surfactants, fatty alcohols and their esters, preferably alcoxylated fatty alcohols, and mixtures thereof, particularly preferably selected from the group consisting of unpolar substances, preferably selected from the group consisting of polyacrylates, polymethacrylates, polyethers, polyesters, polyamides, polynitriles, etherificated celluloses, celluloseacetate, polynitrile, etherificated sugars and mixtures thereof.

The term “phenolic compound” as used herein refers to a compound comprising one or more aromatic hydrocarbon group or groups bound directly to one or more hydroxyl groups (—OH).

The term “carbonyl compound” as used herein refers to a compound comprising one or more carbon atom or atoms, which is/are bound to an oxygen atom via a double bond (C═O).

The term “sulfonating agent” as used herein refers to a compound comprising one or more sulfonic acid functional group or groups and/or to one or more salt(s) thereof, such as sodium or potassium salts and mixtures thereof, which can thus serve as an educt in a sulfonation reaction.

The term “urea derivative” as used herein refers to any derivative of urea, preferably to a compound, which can be formed as a product of a chemical reaction involving urea as a precursor molecule.

Preferably, the condensation product or a salt thereof is produced by a method comprising the steps of
providing component a1)
providing component a2)
optionally: providing component a3) and/or component a4),
mixing components a1) and a2) by stirring, and applying a temperature of at most 105° C.,
stirring the obtained mixture at a temperature in the range of from 100 to 105° C. for a time in the range of from 0.5 to 5 hours
optionally: adding component a3) and/or component a4).

It is preferred that for the condensation product or salt thereof or pharmaceutical composition as defined herein component a1) is selected from the group consisting of phenol and dihydroxydiphenylsulfone.

Additionally or alternatively, it is preferred that component a2) is selected from the group consisting of formaldehyde, acetaldehyde and propionaldehyde.

Additionally or alternatively, it is preferred that component a3), if present, is sulfuric acid, preferably concentrated sulfuric acid.

Additionally or alternatively, it is preferred that component a4), if present, is selected from the group consisting of urea, melamine, the compound according to formula (I), the compound according to formula (II) and the compound according to formula (III),

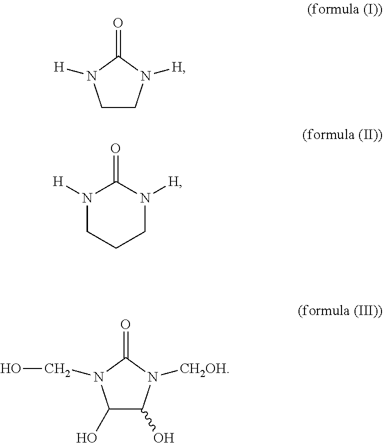

The term “concentrated sulphuric acid” as used herein refers to sulphuric acid in a liquid aggregate state and with a concentration of 90% or more, preferably 93% or more, more preferably 95% or more, even more preferably 97% or more, most preferably 99% or more.

It is further preferred that component a1) is phenol.

Additionally or alternatively component a2) is formaldehyde.

Additionally or alternatively component a3) is present and is sulfuric acid, preferably concentrated sulfuric acid.

Additionally or alternatively component a4) is present and is urea.

Particularly, it is preferred that component a1) is phenol, component a2) is formaldehyde, component a3) is present and is sulfuric acid, preferably concentrated sulfuric acid, and component a4) is present and is urea.

It is preferred that the condensation product or salt thereof is or comprises a compound according to formula (IV), or is or comprises a salt of a compound according to formula (IV)

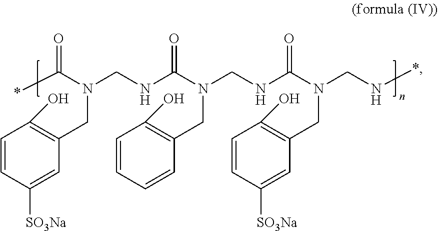

wherein n is an integer in a range of from 1 to 30, preferably in a range of from 2 to 25, particularly preferably in a range of from 3 to 20, further preferably in a range of from 4 to 15, more preferably in a range of from 5 to 12, especially preferably in a range of from 6 to 10, even further preferably in a range of from 7 to 9, or a derivative or pharmaceutically acceptable salt thereof.

Preferably in the prevention and/or treatment of COVID-19 and/or the prevention and/or 15 treatment of a SARS-CoV-2 infection, the condensation product or the one or more salt(s) thereof ortho condensation product and the one or more salt(s) thereof is/are administered at a daily dose in a range of from 0.005 to 10 mg/kg body weight of the subject, preferably in a range of from 0.0075 to 7.5 mg/kg body weight of the subject, preferably 0.01 to 5 mg/kg body weight of the subject, preferably in a range of from 0.025 to 2.5 mg/kg body weight of the subject, particularly preferably in a range of from 0.05 to 2 mg/kg body weight of the subject, especially preferably in a range of from 0.15 to 1 mg/kg body weight of the subject.

Preferably, the daily dose, as described herein, refers to the total dose of all condensation products according to the invention and the salts thereof, as far as present.

Particularly preferably, the term “condensation product”, as used herein, describes the entirety of all present condensation products according to the invention, where applicable. Thus, in case a parameter is to be determined for the condensation product, it is preferred that the entirety of all present condensation products are considered and included in the determination of said parameter. I.e. in case a daily dose of administration is to be determined, the dose is determined based on the entirety of all administered condensation products according to the invention.

Particularly preferably, in case a parameter is to be determined for the condensation product, it is preferred that the entirety of all present condensation products and the entirety of their present salts, as far as present, are considered and included in the determination of said parameter. I.e. in case a daily dose of administration is to be determined, the dose is determined based on the entirety of all administered condensation products according to the invention and all administered salts of condensation products according to the invention, as far as present.

Likewise, in case a condensation product is administered, the term “condensation product”, as used herein, preferably includes the entirety of all present condensation products according to the invention, particularly preferably the entirety of all present condensation products and the entirety of their present salts.

The term “daily dose” as used herein refers to an average daily amount per body weight of the condensation product as described herein or a salt thereof or the condensation product and a salt thereof, which is administered to a subject and does not necessarily require the administration to be performed every single day of a given time period. If the administration of the condensation product or a salt thereof or the condensation product and a salt thereof is performed once or more in a given number of days, the daily dose can be calculated by dividing the total administered amount of the condensation product or a salt thereof or the condensation product and a salt thereof per body weight of the subject by the respective given number of days.

Furthermore and preferably, the given number of days only describe a part of the treatments and/or preventions as described herein. Thus, in a first part of the treatments and/or preventions (i.e. a first given number of days), the daily dose may be different from a second, third, fourth, fifth or further part of the treatments and/or preventions (i.e. a second, third, fourth, fifth or further given number of days). Additionally or alternatively, the given number of days of the first, second, third, fourth, fifth and/or further part of the treatments and/or preventions may be different from the other part(s) of the treatments and/or preventions.

It is thus preferred, that the daily dose is higher in a first part of the treatment of COVID-19 and/or SARS-CoV-2, than in a second or further part.

It is thus preferred, that the daily dose is lower in a first part of the treatment of COVID-19 and/or SARS-CoV-2, than in a second or further part.

It is thus preferred, that the daily dose is higher in a first part of the prevention of COVID-19 and/or SARS-CoV-2, than in a second or further part.

It is thus preferred, that the daily dose is lower in a first part of the prevention of COVID-19 and/or SARS-CoV-2, than in a second or further part.

Furthermore, it is preferred that the prevention and/or treatment of COVID-19 and/or the prevention and/or treatment of a SARS-CoV-2 infection further comprises the step of administering one or more antiviral agent(s),
preferably, the, one or more or all antiviral agent(s) has/have an antiviral activity, preferably a virostatic or virucidal activity, against SARS-CoV-2 and preferably the, one or more or all antiviral agent(s) target(s) a surface glycoprotein, preferably the spike protein, of SARS-CoV-2,
preferably the, one or more or all antiviral agent(s) is/are selected from the group consisting of Camostat, Remdesivir, Casirivimab, Imdevimab, Regdanvimab, Bamlanivimab, Etesevimab, Sotrovimab and mixtures thereof.

For instance, the antiviral agent is a mixture comprising Casirivimab and Imdevimab or a mixture comprising Bamlanivimab and Etesevimab.

The term “antiviral activity” as used herein refers to any activity, which reduces the entrance into a host cell, the replication, the spread, the infectivity, the expression of genes, the translation of mRNA, the packaging of proteins of a virus.

The term “targeting a surface glycoprotein” as used herein refers to the target of an agent having an antiviral activity, wherein the target is a surface glycoprotein of the virus. Thus, a viral agent targeting a surface glycoprotein binds to or affects, preferably negatively affects, in any way the surface glycoprotein.

The term “surface glycoprotein” refers to any glycoprotein, which is present on the surface of a virus. Preferably, such a surface glycoprotein is the spike-protein, which is also referred to as S-glycoprotein of SARS-CoV-2.

It is further preferred that the pharmaceutical composition further comprises one or more antiviral agent(s). Additionally or alternatively, the, one or more or all of the antiviral agent(s) has/have an antiviral activity, preferably a virostatic or virucidal activity, against SARS-CoV-2,
preferably, the, one or more or all of the antiviral agent(s) target(s) a surface glycoprotein, preferably the spike protein, of SARS-CoV-2,
preferably, the, one or more or all of the antiviral agent(s) is/are selected from the group consisting of Camostat, Remdesivir, Casirivimab, Imdevimab, Regdanvimab, Bamlanivimab, Etesevimab, Sotrovimab and mixtures thereof.

For instance, the antiviral agent is a mixture comprising Casirivimab and Imdevimab or a mixture comprising Bamlanivimab and Etesevimab.

Moreover, the present invention relates to a pharmaceutical set comprising
(i) a pharmaceutical composition as defined herein and
(ii) one or more antiviral agent(s), preferably wherein the, one or more or all of the antiviral agent(s) has/have an antiviral activity, preferably a virostatic or virucidal activity, against SARS-CoV-2 and preferably wherein the, one or more or all of the antiviral agent(s) target(s) a surface glycoprotein, preferably the spike protein, of SARS-CoV-2,
   preferably wherein the, one or more or all of the antiviral agent(s) is/are selected from the group consisting of Camostat, Remdesivir, Casirivimab, Imdevimab, Regdanvimab, Bamlanivimab, Etesevimab, Sotrovimab and mixtures thereof,
   particularly preferably, the antiviral agent is a mixture comprising Casirivimab and Imdevimab or a mixture comprising Bamlanivimab and Etesevimab,
for use in a method for the prevention or treatment of COVID-19 and/or for the prevention or treatment of a SARS-CoV-2 infection,
wherein the method comprises the step of administering the condensation product or a salt thereof or the condensation product and a salt thereof to a subject, preferably a human,
wherein the prevention of COVID-19 comprises the step of administering to a subject the condensation product or a salt thereof ortho condensation product and a salt thereof before an infection with SARS-CoV-2, and
wherein the treatment of COVID-19 comprises the step of administering to a subject the condensation product or a salt thereof or the condensation product and a salt thereof, wherein a first administration is performed not later than 3 months after a positive diagnosis of the SARS-CoV-2 infection of a subject, and
wherein the treatment of a SARS-CoV-2 infection comprises the step of administering to a subject the condensation product or a salt thereof or the condensation product and a salt thereof, wherein a first administration is performed not later than 3 months after a positive diagnosis of the SARS-CoV-2 infection of a subject.

What is said herein in the context of the condensation product or a salt thereof or, respectively, the pharmaceutical composition, applies accordingly for the pharmaceutical set, where applicable.

Preferably, components (i) and (ii) are present in a form separated from each other in the pharmaceutical set. Therefore, components (i) and (ii) may be present in separate containments, such as vials, tubes, bottles, cups, boxes or bags.

Preferably, a pharmaceutical set, as described herein, may further comprise one or more additional components, preferably selected from the group consisting of syringes; disinfection wipes; files; pipettes; containers, preferably dosing aerosol containers; dispensers, preferably dispensers for spraying, particularly preferably spray bottles; pens; pads; tapes; foils; stickers; wraps; lamps and polishes.

It is preferred in the condensation product or salt thereof or pharmaceutical composition or pharmaceutical set as described herein that the condensation or salt thereof product or pharmaceutical composition is present in a form selected from the group consisting of pills, tablets, lozenges, granules, capsules, preferably hard or soft gelatine capsules, aqueous solutions, alcoholic solutions, oily solutions, syrups, emulsions, suspensions, suppositories, pastilles, solutions for injection or infusion, ointments, tinctures, creams, lotions, powders, sprays, transdermal therapeutic systems, nasal sprays, aerosols, aerosol mixtures, microcapsules, implants, rods, patches and gels.

Particularly preferably, the condensation product or salt thereof or pharmaceutical composition is present in a form selected from the group consisting of aqueous solutions or sprays

Especially preferably, the condensation product or salt thereof or pharmaceutical composition is present as an oral or nasal spray or a mouthwash.

With regard to a treatment and/or prevention as described herein, it is preferred that the administration is performed at an administration scheme of at least once in 7 days, preferably at least once in 6 days, preferably at least once in 5 days, preferably at least once in 4 days, preferably at least once in 3 days, preferably at least once in 2 days, preferably at least once a day, preferably at least twice a day, preferably at least three times a day, preferably at least four times a day, preferably at least five times a day.

Preferably, the administration scheme is applied over a time period of at least 1 week, preferably at least 2 weeks, preferably at least 3 weeks, preferably at least 4 weeks, preferably at least 6 weeks, preferably at least 2 months, preferably at least 3 months, preferably at least 4 months, preferably at least 5 months, preferably at least 6 months.

Applying the administration scheme for a certain time period as described herein and within a certain regularity as described herein are not mutually exclusive. E.g., an administration of at least once in 7 days for at least 4 weeks describes the regularity of once in 7 days for a duration of at least 4 weeks or longer. The term “once in 7 days” as used herein is to be understood as one administration within a time frame of 7 days, wherein the administration may be at the beginning, the end or at any other time point within the 7 days. In case the administration is repeated, i.e. the time period of the administration scheme is longer than the administration interval, the time point of the respective administration (the beginning, the end or at any other time point within the interval) may vary between the respective administration intervals. E.g. in an administration of at least once in 7 days for at least 4 weeks, the administration in the first 7 days (first interval) may be performed at the beginning (day 1) of the interval and the administration in the second 7 days (second interval) may be performed on day 3 of the interval. The same applies accordingly to any other regularity and time period as described herein.

Preferably, the administration scheme in a first part of the treatments and/or preventions as described herein, the daily dose may be different from a second, third, fourth, fifth or further part of the treatments and/or preventions as described herein. E.g., the administration scheme in a first part of the treatments and/or preventions may be at least once a day, wherein in a second or further part of the treatments and/or preventions, the administration scheme may be once in two days.

It is thus preferred that the frequency of administration is higher in a first part of the treatment of COVID-19 and/or SARS-CoV-2, than in a second or further part.

It is thus preferred that the frequency of administration is lower in a first part of the treatment of COVID-19 and/or SARS-CoV-2, than in a second or further part.

It is thus preferred that the frequency of administration is higher in a first part of the prevention of COVID-19 and/or SARS-CoV-2, than in a second or further part.

It is thus preferred that the frequency of administration is lower in a first part of the prevention of COVID-19 and/or SARS-CoV-2, than in a second or further part.

Moreover, it is preferred that the step of administering is performed by or comprises an administration via an administration route selected from enteral or parenteral.

Preferably, the step of administering is performed by or comprises an administration via an administration route selected from the group consisting of oral, nasal, intravenous, intraarterial, inhalation and absorption over a mucous membrane.

The present invention further relates to a pharmaceutical composition or pharmaceutical set as described herein. Therefore, the present invention relates to a pharmaceutical composition or pharmaceutical set comprising
(i) a condensation product as defined herein or a salt thereof or the condensation product and a salt thereof, and
(ii) one or more antiviral agents, wherein the, one or more or all of the antiviral agent(s) has/have an antiviral activity, preferably a virostatic or virucidal activity, against SARS-CoV-2 and preferably target(s) a surface glycoprotein, preferably the spike protein, of SARS-CoV-2,
   preferably, the, one or more or all of the antiviral agent(s) is/are selected from the group consisting of Camostat, Remdesivir, Casirivimab, Imdevimab, Regdanvimab, Bamlanivimab, Etesevimab, Sotrovimab and mixtures thereof.

For instance, the antiviral agent is a mixture comprising Casirivimab and Imdevimab or a mixture comprising Bamlanivimab and Etesevimab.

What was said herein with regard to the pharmaceutical composition or the pharmaceutical set for use as described herein applies accordingly to the pharmaceutical composition or the pharmaceutical set independent of its use, where applicable.

It is preferred that the pharmaceutical composition or one or more or all components of the pharmaceutical set is/are present in a form selected from the group consisting of pills, tablets, lozenges, granules, capsules, preferably hard or soft gelatine capsules, aqueous solutions, alcoholic solutions, oily solutions, syrups, emulsions, suspensions, suppositories, pastilles, solutions for injection or infusion, ointments, tinctures, creams, lotions, powders, sprays, transdermal therapeutic systems, nasal sprays, aerosols, aerosol mixtures, microcapsules, implants, rods, patches and gels.

Preferably, the pharmaceutical composition or one or more or all components of the pharmaceutical set is/are present in a form selected from the group consisting of aqueous solutions or sprays.

Preferably, the pharmaceutical composition or one or more or all components of the pharmaceutical set is/are an oral or nasal spray or a mouthwash.

The invention further relates to a pharmaceutical composition comprising a condensation product as defined herein or a salt thereof or the condensation product and a salt thereof, wherein the pharmaceutical composition is an oral or nasal spray or a mouthwash.

Furthermore, the invention further relates to a pharmaceutical set comprising a pharmaceutical composition, wherein the pharmaceutical composition is an oral or nasal spray or a mouthwash.

Moreover, the invention relates to a method for the prevention or treatment of COVID-19 and/or for the prevention or treatment of a SARS-CoV-2 infection, wherein the method comprises the step of administering a condensation product or a salt thereof or the condensation product and a salt thereof or a pharmaceutical composition as described herein.

What was said with regard to the condensation product or a salt thereof or the pharmaceutical composition for use in a method for the prevention or treatment of COVID-19 and/or for the prevention or treatment of a SARS-CoV-2 infection applies accordingly to the method for the prevention or treatment of COVID-1 and/or for the prevention or treatment of a SARS-CoV-2 infection. Particularly, the preferred features of the condensation product or a salt thereof or the pharmaceutical composition for such use are also preferred features in the method for the prevention or treatment of COVID-19 and/or for the prevention or treatment of a SARS-CoV-2 infection.

Further aspects and advantages of the invention result from the subsequent description of preferred examples.

### EXAMPLES

### Example 1: Producing a Condensation Product According to the Invention

Solutions are understood as meaning aqueous solutions if not expressly specified otherwise. ppm relates to parts by weight.

The molecular weight determinations are carried out using gel permeation chromatography (GPC):

Stationary phase: poly(2-hydroxymethacrylate) gel crosslinked with ethylene glycol dimethacrylate, obtainable commercially as HEMA BIO from PSS, Mainz, Germany.
Eluent: mixture of 30% by weight of tetrahydrofuran (THF), 10% by weight of acrylonitrile, 60% by weight of 1 molar NaNO3 solution
Internal standard: 0.001% by weight of benzophenone, based on eluent
Flow: 1.5 ml/min
Concentration: 1% by weight in the eluent containing internal standard
Detection: UV/V is spectrometrically at 254 nm
Calibration using polystyrene calibration part from PSS.
Mn: number-average molecular weight in [g/mol]
Mw: weight-average molecular weight in [g/mol]

For the determination of free formaldehyde, a flow injection apparatus according to Huber is employed, see Fresenius Z. Anal. Chem. 1981, 309, 389. The column chosen is a thermostated reaction column 170×10 mm, filled with glass beads, which is operated at 75° C. The detector (continuous flow detector) is set at a wavelength of 412 nm. The procedure is as follows: For the preparation of a reagent solution, 62.5 g of ammonium acetate are dissolved in 500 ml of distilled water, 7.5 ml of concentrated acetic acid and 5.0 ml of acetylacetone are added and filled up to 1000 ml with distilled water. 0.1 g of the condensation product to be investigated is weighed into a 10 ml volumetric flask, filled up to 10 ml with distilled water and the respective sample solution is obtained. 100 μl of sample solution in each case are added, mixed with reagent solution and a mean residence time of 1.5 minutes is set, which corresponds to a flow of 35 ml/min.

For the determination of the absolute values, the flow injection apparatus is calibrated with formaldehyde solutions of known content.

### Example 1.1: Condensation Product A

Reactants were:
a1) phenol,
a2) concentrated sulfuric acid,
a3) formaldehyde,
a4) urea

2.04 kg of phenol are introduced into a stirring apparatus and treated with 2.48 kg of concentrated sulfuric acid (96% by weight) for 20 minutes. Care is to be taken here that the temperature does not exceed 105° C. Subsequently, the reaction mixture is stirred at 100 to 105° C. for 2 hours and then diluted with 0.34 kg of water of 20° C. and cooled to 70° C.

2.06 kg of aqueous urea solution (68% by weight) are metered in, the temperature rising to 95° C.; subsequently the mixture is cooled to 75° C.

4.10 kg of aqueous formaldehyde solution (30% by weight) are added over a period of 90 minutes, care being taken that the temperature does not rise above 75° C.

Subsequently, it is partially neutralized using 0.78 kg of aqueous sodium hydroxide solution (50% by weight), 0.30 kg of water are added, and the mixture is subsequently stirred for 30 minutes and cooled further.

1.36 kg of phenol are added at a temperature of 50° C. 1.14 kg of aqueous formaldehyde solution (30% by weight) are subsequently metered in at 50° C. over 20 minutes and the mixture is subsequently stirred for a further 30 minutes at 55° C.

The final adjustment of concentration and pH is carried out by addition of 1.40 kg of sodium hydroxide solution (50% by weight) and 2.5 kg of water. 18.5 kg of reaction solution 1.1 are obtained containing 43% by weight of non-volatile fractions.

The analysis of the reaction solution affords the following values:
sodium sulfate by IC (based on non-volatile fractions): 6.8% by weight;
phenol by HPLC (based on non-volatile fractions): 0.36% by weight;
4-phenolsulfonic acid by HPLC (based on non-volatile fractions): 2.89% by weight;
free formaldehyde: 75 ppm, based on non-volatile fractions;
Mn 890 g/mol, M_{w} 7820 g/mol, determined by GPC.

### Example 1.2: Condensation Product B

Reactants were
a) phenol,
b) concentrated sulfuric acid,
c) formaldehyde,
d) urea

2.04 kg of phenol are introduced into a stirring apparatus and treated with 2.48 kg of concentrated sulfuric acid (96% by weight) for 20 minutes. Care is to be taken here that the temperature does not exceed 105° C. Subsequently, the reaction mixture is stirred at 100 to 105° C. for 2 hours and then diluted with 340 g of water.

2.05 kg of urea solution (68% by weight) are metered in, care being taken that the temperature does not exceed 95° C.

3.60 kg of aqueous formaldehyde solution (30% by weight) are then added at 83 to 93° C. over a period of 1.5 hours.

After a stirring time of 15 minutes, 800 g of aqueous sodium hydroxide solution (50% by weight) are added, care being taken that the temperature does not exceed 85° C., so that the pH is subsequently between 7.3 and 7.5. 11.3 kg of reaction solution 1.2 containing 47% by weight of non-volatile fractions are obtained.

The analysis of reaction solution affords the following values:
sodium sulfate by IC (based on non-volatile fractions): 10.3% by weight;
phenol by HPLC (based on non-volatile fractions): 0.74% by weight;
4-phenolsulfonic acid by HPLC (based on non-volatile fractions): 1.36% by weight;
free formaldehyde: 99 ppm, based on non-volatile fractions;
Mn 1990 g/mol, Mw 17.020 g/mol, determined by GPC.

### Example 2: Formulation Examples

| Phase | Material | Amount [wt/v-%] |
|---|---|---|
| A | Kolliphor P 188 | 0-1.5 |
| | Kolliphor P 407 | 15-18 |
| | Sterile water | 50 |
| B | Hyaluronic acid | 0.5-1 |
| | NaCl | 0.9 |
| | Condensation product | 0.01/0.025/0.05 |
| | according to one of Examples 1.1 or 1.2 | |
| | Benzalkonium chloride | 0.01 |
| C | Sterile water | ad 100 ml |

The amount of NaCl slightly varied, depending on the osmolarity.

Furthermore, the amount of the condensation product according to one of Examples 1.1, or 1.2 varied (as indicated), depending on the desired amount of condensation product.

For preparation of the above-outlined formulation, components of phase A were combined in a beaker, mixed together, and refrigerated overnight. Subsequently, components of phase B were added to phase A and the mixture was slowly mixed using an overhead mixer. Finally, the overall volume of the mixture was adjusted to 100 ml using sterile water (phase C).

Upon preparation, the formulation was provided either as a nasal spray or a mouthwash solution.

### Example 3: Antiviral Activity

### Example 3.1: Cell Culture Treated with the Condensation Product

A condensation product was produced according to Example 1.2.

Two different SARS-CoV-2 isolates were obtained (isolate 1 and isolate 2).

Cells were cultured and infected with isolate 1 or isolate 2 with an MOI of 0.001 or, respectively, with an MOI of 0.01. The cells were subsequently treated with either 1 μg/ml, 10 μg/ml or 100 μg/ml of the condensation product or, respectively, with water as a control.

The viral titer was then determined. It was surprisingly found that the condensation product according to the invention was able to reduce the viral titer for both SARS-CoV-2 isolates in a dose-dependent manner.

### Example 3.2: Cell Culture Treated with a Formulation Comprising the Condensation Product

The formulation according to Example 2 was provided, however with different concentrations of the condensation product:
A-1: 1,000 μg/ml
A-2: 500 μg/ml
A-3: 250 μg/ml
A-4: 100 μg/ml
A-5: 50 μg/ml
A-6: 0 μg/ml

Calu3 cells were cultured and infected with SARS-CoV-2. The cells were subsequently treated with one of the prepared formulations (one of A-1 to A-6), with 100 μM Camostat (control), with 10 μM Camostat (control) or, respectively, with a water (control).

The viral titer was then determined. It was surprisingly found that the condensation product according to the invention was able to reduce the viral titer also when being prepared in a formulation. Furthermore, the reduction was similar to the reduction observed for Camostat; however, the condensation product was used in a much lower concentration.

### Example 3.3: Inhibition of SARS-CoV-2 Replication

A condensation product was produced according to Example 1.2.

Cells were infected with SARS-CoV-2 and treated with the condensation product in different concentrations. The inhibition of viral replication was subsequently determined.

It was surprisingly found that the condensation product according to the invention inhibited the viral replication of SARS-CoV-2 in a dose-dependent manner. Already at a concentration of 1 μM, an inhibition of the viral replication was obtained.

### Example 3.4: Precision Cut Human Lung Slices

A condensation product was produced according to Example 1.2.

Precision cut human lung slices were provided and infected with SARS-CoV-2. Subsequently, the slices were treated with 1 μg/ml, 10 μg/ml or 100 μg/ml of the condensation product or with 1 μg/ml, 10 μg/ml or 100 μg/ml of Camostat (control) or with water as a control.

The viral titer was then determined. Both, Camostat and the condensation product according to the invention reduced the viral titer in a dose-dependent manner.

However, it was surprisingly found that the condensation product according to the invention provided a stronger reduction of the viral titer when compared to the respective concentration of Camostat.

1. A condensation product or salt thereof,
wherein the condensation product is obtained by a reaction of
a1) phenol, and
a2) formaldehyde, and
a3) sulfuric acid, and
a4) urea,
wherein the condensation product or salt thereof has a molecular weight M_{w} in the range of from 500 g/mol to 50,000 g/mol.

2. The condensation product or salt thereof according to claim 1, wherein the condensation product or salt thereof comprises a compound according to formula (IV), and/or a salt of the compound according to formula (IV) 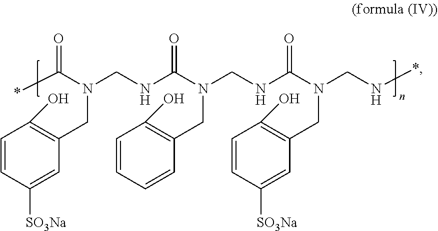
wherein n is an integer in a range of from 1 to 30,
or a derivative or pharmaceutically acceptable salt thereof.

3. The method according to claim 14, wherein the condensation product and/or the salt thereof is administered at a daily dose in a range of from 0.005 to 10 mg/kg body weight of the subject.

4. The method according to claim 14,
wherein the prevention and/or treatment of COVID-19 and/or the prevention and/or treatment of a SARS-CoV-2 infection further comprises administering one or more antiviral agent,
wherein the one or more of the antiviral agent has a virostatic or virucidal activity against SARS-CoV-2 and wherein the one or more antiviral agent targets a surface glycoprotein, of SARS-CoV-2.

5. A pharmaceutical composition comprising the condensation product or salt thereof according to claim 1 and one or more antiviral agent.

6. (canceled)

7. The pharmaceutical composition according to claim 5, wherein the composition is present in a form selected from the group consisting of pills, tablets, lozenges, granules, capsules, preferably hard or soft gelatine capsules, aqueous solutions, alcoholic solutions, oily solutions, syrups, emulsions, suspensions, suppositories, pastilles, solutions for injection or infusion, ointments, tinctures, creams, lotions, powders, sprays, transdermal therapeutic systems, nasal sprays, aerosols, aerosol mixtures, microcapsules, implants, rods, patches, and gels.

8. The method according to claim 14, wherein the administering is performed at an administration scheme of at least once in 7 days,
and wherein the administration scheme is applied over a time period of at least 1 week.

9. The method according to claim 14, wherein the administering is performed by administration via a route selected from the group consisting of oral, nasal, intravenous, intraarterial, inhalation, and absorption over a mucous membrane.

10. A pharmaceutical composition set comprising
(i) a condensation product as defined in claim 1 and/or a salt thereof, and
(ii) one or more antiviral agent
selected from the group consisting of Camostat, Remdesivir, Casirivimab, Imdevimab, Regdanvimab, Bamlanivimab, Etesevimab, Sotrovimab, and mixtures thereof.

11. The pharmaceutical composition according to claim 10, wherein the pharmaceutical composition is in a form selected from the group consisting of pills, tablets, lozenges, granules, capsules, aqueous solutions, alcoholic solutions, oily solutions, syrups, emulsions, suspensions, suppositories, pastilles, solutions for injection or infusion, ointments, tinctures, creams, lotions, powders, sprays, transdermal therapeutic systems, nasal sprays, aerosols, aerosol mixtures, microcapsules, implants, rods, patches, and gels.

12. The pharmaceutical composition according to claim 11, wherein the pharmaceutical composition is an oral spray, nasal spray, or a mouthwash.

13. (canceled)

14. A method for preventing or treating COVID-19 and/or preventing or treating SARS-CoV-2 infection comprising
administering the condensation product and/or a salt thereof according to claim 1 to a subject,
wherein prevention of COVID-19 comprises administering to the subject the condensation product and/or a salt thereof, wherein a first administration is performed not later than 3 months after a positive diagnosis of the SARS-CoV-2 infection of the subject, and
wherein treatment of COVID-19 comprises administering to the subject the condensation product and/or a salt thereof, wherein a first administration is performed not later than 3 months after a positive diagnosis of the SARS-CoV-2 infection of the subject, and
wherein treatment of a SARS-CoV-2 infection comprises administering to the subject the condensation product and/or a salt thereof, wherein a first administration is performed not later than 3 months after a positive diagnosis of the SARS-CoV-2 infection of the subject,
optionally wherein the condensation product or the salt thereof further comprises one or more pharmaceutically acceptable carrier.

15. The method according to claim 4 wherein the one or more antiviral agent is selected from the group consisting of Camostat, Remdesivir, Casirivimab, Imdevimab, Regdanvimab, Bamlanivimab, Etesevimab, Sotrovimab, w and mixtures thereof.
